(19) United States
(12) Patent Application Publication
(10) Pub. No.: US 2026/0138960 A1
(43) Pub. Date: May 21, 2026

(54) A PROCESS FOR THE PREPARATION OF CRYSTALLINE FORM OF TAFAMIDIS

(71) Applicant: BIOPHORE INDIA PHARMACEUTICALS PVT. LTD, Hyderabad (IN)

(72) Inventors: Manik Reddy PULLAGURLA, Hyderabad (IN); Jagadeesh Babu RANGISETTY, Hyderabad (IN); Bhaskar Reddy PITTA, Hyderabad (IN); Kiran Kumar KOTHAKONDA, Hyderabad (IN); Rajesham BOGE, Hyderabad (IN); Srinivas ARUTLA, Hyderabad (IN)

(21) Appl. No.: 19/121,967

(22) PCT Filed: Oct. 14, 2023
(86) PCT No.: PCT/IB2023/060380

§ 371 Date: Apr. 17, 2025

(30) Foreign Application Priority Data
Oct. 17, 2022 (IN) 202241059152
Sep. 13, 2023 (IN) 202341061587

(51) Int. Cl. C07D 263/57

(52) U.S. Cl. CPC C07D 263/57

(57) ABSTRACT

The present invention relates to a stable crystalline form of Tafamidis having characteristic 2-theta peaks at 13.6±0.2, 20.4±0.2 and 27.5±0.2. The present invention provides Tafamidis having particle size (d₉₀) less than 100 microns. Furthermore, the present invention relates to a novel process for the preparation of crystalline form of Tafamidis (1) with particle size (d₉₀) less than 100 microns and specific surface area less than 30 sq·m/g. The present invention relates to a stable oral dosage form comprising composition containing Tafamidis, particularly, a soft gel capsule comprising composition containing Tafamidis and to methods for their preparation. The present invention relates to a stable oral dosage form comprising composition containing Tafamidis which is indicated for the treatment of the cardiomyopathy of wild type or hereditary transthyretin-mediated amyloidosis in adults to reduce cardiovascular mortality and cardiovascular-related hospitalization.

### FIELD OF INVENTION

The present invention relates to Tafamidis having particle size (d₉₀) less than 100 microns. Further, the present invention relates to a novel process for the preparation of Tafamidis with particle size (d₉₀) less than 100 microns and specific surface area less than 30 sq·m/g. Additionally, the present invention further provides process for the preparation of Tafamidis solvated forms.

The invention relates to a stable oral dosage form comprising composition containing Tafamidis. Particularly, the invention relates to a soft gel capsule comprising composition containing Tafamidis and to methods for their preparation.

The invention relates to a stable oral dosage form comprising composition containing Tafamidis, particularly, a soft gel capsule comprising composition containing Tafamidis Form 6 and to methods for their preparation.

The stable dosage form comprising composition containing Tafamidis Form 6 is indicated for the treatment of the cardiomyopathy of wild type or hereditary transthyretin-mediated amyloidosis in adults to reduce cardiovascular mortality and cardiovascular-related hospitalization.

### BACKGROUND OF THE INVENTION

Tafamidis is benzoxazole derivative chemically known as 2-(3,5-dichlorophenyl)-1,3-benzoxazole-6-carboxylic acid. Tafamidis has a molecular weight of 308.12 g/mol. Tafamidis free acid structurally as shown below.

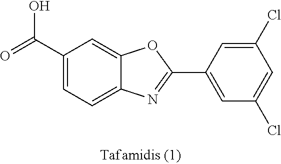 [SMILES: O=C(O)c1ccc2nc(-c3cc(Cl)cc(Cl)c3)oc2c1]

Synthetic routes for 6-carboxy-2-(3,5-dichlorophenyl)-benzoxazole (hereinafter “the compound of Formula I”) are described in U.S. Pat. No. 7,214,695 and solid forms of the meglumine salt of the compound of Formula I are described in U.S. patent application Ser. No. 14/345,111, which is the U.S. national phase of International Application No. PCT/IB2012/054748, all of which are hereby incorporated herein by reference in their entireties for all purposes, and has the structure shown below.

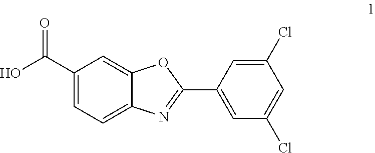 [SMILES: O=C(O)c1ccc2nc(-c3cc(Cl)cc(Cl)c3)oc2c1]

The compound of Formula I stabilizes the protein transthyretin (TTR), dissociation of which is implicated in TTR amyloidosis (i.e., the compound of Formula I prevents dissociation of the native TTR tetramer into monomers, which results in the inhibition of TTR amyloid fibril formation) and is being developed for use in the treatment of transthyretin amyloid diseases.

U.S. Pat. No. 9,770,441 of Pfizer Inc discloses different polymorphic forms of Tafamidis (1) as form 1, form 2, form 4, form 6 and amorphous form. and its preparation involves the reaction of 4-amino-3-hydroxybenzoic acid with 3,5-dichlorobenzoylchloride to provide crude 4-[(3,5-dichlorobenzoyl) amino]-3-hydroxybenzoic acid which is further isolated after work up using triethylamine. The obtained compound further cyclized to form Tafamidis and converted to form 1 using isopropanol. Certain solid forms may also exhibit enhanced stability or may be more readily manufactured in high purity in large quantities, and thus are more suitable for inclusion in pharmaceutical formulations. U.S. Pat. No. 9,770,441 claims Form 1 wherein said form (i) has a powder X-ray diffraction pattern comprising peak at diffraction angles (2θ) of 28.6±0.2; (ii) has a NMR spectrum comprising 13C chemical shifts (ppm) at 120.8±0.2 and 127.7±0.2 and (iii) Raman shift peaks (cm⁻¹) at 1292±2 and 1615±2.

WO/2020/232325 of Teva's application discloses preparation of Tafamidis amorphous form and crystalline form I, II, III, IV and V. Tafamidis form I is obtained from tetrahydrofuran (THF), form II from 2-methyl tetrahydrofuran, form III, and form IV from acetic acid at different temperatures. Tafamidis form Vis obtained using tetrahydrofuran and methanol as antisolvent.

WO/2021/019448 of Honour lab application discloses different solid dispersion forms of Tafamidis (1). Wherein Tafamidis solid dispersions are disclosed with co povidone and hydroxypropyl methylcellulose.

Tafamidis and Tafamidis meglumine are available as soft gelatin capsule form in United States, Europe and other markets. Tafamidis is marketed as Vyndamax®, while Tafamidis meglumine is marketed as Vyndaqel® in USA. However, it is marketed under Vyndagel® (Tafamidis & Tafamidis meglumine) in Europe. VYNDAMAX is indicated for the treatment of cardiomyopathy of wild type or hereditary transthyretin-mediated amyloidosis in adults to reduce cardiovascular mortality and cardiovascular-related hospitalization.

Tafamidis meglumine and Tafamidis are biopharmaceutical classification system (BCS) class II drugs, which means that they are poorly soluble, but permeate biological membranes well.

According to the Vyndaqel® European Public assessment report “[t]he chosen excipients in the capsule fill solution are: polyethylene glycol (suspending vehicle), sorbitan monoleate (surfactant/wetting agent), polysorbate 80 (surfactant/wetting agent),” while “[t]he excipients in the capsule shell are: gelatin, propylene glycol, purified water, ‘Sorbitol special-glycerin blend’ (d-sorbitol, 1,4 sorbitan, mannitol and glycerin) and titanium dioxide (E 171) and black printing ink.”

U.S. Pat. No. 11,523,993 claims a tablet composition comprising a) Tafamidis or Tafamidis meglumine; b) at least one acidifier, wherein the at least one acidifier is present in an amount of from about 0.1% w/w to about 10% w/w based on the total weight of the composition; and c) at least one pharmaceutically acceptable excipient; wherein the tablet comprises 61 mg of Tafamidis or 20 mg of Tafamidis meglumine.

Still, there is a significant need for the development of novel process for the preparation of crystalline form of Tafamidis, which is stable during pharmaceutical formulations. Further, there is an unmet need to understand the challenges associated with development of stable oral dosage forms like soft gel capsules comprising crystalline form of Tafamidis composition and methods for their preparation.

### OBJECTIVE OF THE INVENTION

The present invention relates to Tafamidis with particle size (d₉₀) less than 100 microns. Further, the present invention relates to a novel process for the preparation of Tafamidis with particle size (d₉₀) less than 100 microns, bulk density of 0.20 to 0.50 g/ml and specific surface area less than 30 sq·m/g. Additionally, the present invention further provides process for the preparation of Tafamidis solvated forms.

In another objective, the present invention relates to a stable oral dosage form comprising composition containing Tafamidis with particle size (d₉₀) less than 100 microns, bulk density of 0.20 to 0.50 g/ml and specific surface area less than 30 sq·m/g. Particularly, the invention relates to a soft gel capsule comprising composition containing Tafamidis and to methods for their preparation.

In another objective, the present invention relates to a stable oral dosage form comprising composition containing Tafamidis, particularly, a soft gel capsule comprising composition containing Tafamidis Form 6 with particle size (d₉₀) less than 100 microns, bulk density of 0.20 to 0.50 g/ml and specific surface area less than 30 sq·m/g and to methods for their preparation.

### SUMMARY OF THE INVENTION

The present invention relates to Tafamidis with particle size (d₉₀) less than 100 microns. Further, the present invention relates to a novel process for the preparation of crystalline form of Tafamidis with particle size (d₉₀) less than 100 microns and specific surface area less than 30 sq·m/g.

In certain aspects, the present invention provides a process for the preparation of Tafamidis, comprising the steps of:
a. dissolving Tafamidis meglumine (2) in water;
b. adjusting pH of the reaction mass with a suitable acid;
c. adding polar aprotic solvent to the reaction mixture;
d. separating organic layers and adding polar aprotic solvent or mixture of solvents to the organic layer;
e. adding the reaction mixture to suitable solvent.
f. filtering the solid and washing with suitable solvent;
g. milling the wet material at suitable temperature; and
h. drying and isolating Tafamidis

In another aspect of the present invention provides a process for the preparation of Tafamidis, comprising the steps of:
i. providing a mixture of Tafamidis in an organic solvent;
ii. heating the reaction mass at suitable temperature;
iii. adding suitable polar aprotic solvent to the reaction mixture;
iv. adding the reaction mixture to suitable solvent;
v. filtering the solid and washing with suitable solvents
vi. milling the wet material at suitable temperature; and
vii. drying and isolating Tafamidis.

In another aspect, the present invention provides Tafamidis having particle size distribution of D₉₀ less than 100 microns, preferably D₉₀ less than 50 microns.

In another aspect, the present invention provides process for the preparation of solvated forms of Tafamidis.

Yet another aspect, Tafamidis obtained in the present invention is having characteristic X-Ray diffraction peaks at 2 theta values at 13.6±0.2, 20.4±0.2 and 27.5±0.2 (herein after referred as “Form 6”)

In another aspect, the present invention provides a stable solid oral dosage form comprising composition containing Tafamidis, and at least one or more pharmaceutically acceptable excipients, and to methods for their preparation.

In another aspect, the present invention provides a stable solid oral dosage form comprising composition containing Tafamidis having d₉₀ particle size less than 100μ, bulk density of 0.20 to 0.50 g/ml and specific surface area of less than 30 m²/g, and at least one or more pharmaceutically acceptable excipients.

Yet another aspect, the present invention provides a stable soft gel capsule comprising composition containing Tafamidis Form 6, and at least one or more pharmaceutically acceptable excipients, and to methods for their preparation.

In further aspect, the present invention provides a stable solid dosage form composition containing Tafamidis which is indicated for the treatment of the cardiomyopathy of wild type or hereditary transthyretin-mediated amyloidosis in adults to reduce cardiovascular mortality and cardiovascular-related hospitalization.

### DETAILED DESCRIPTION OF THE DRAWINGS

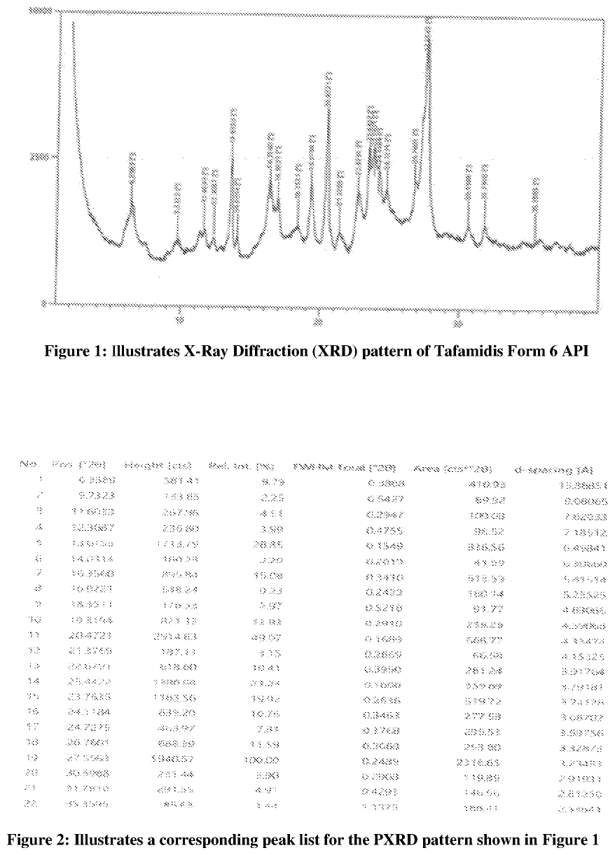

FIG. 1: Illustrates X-Ray Diffraction (XRD) pattern of Tafamidis Form 6 API.

FIG. 2: Illustrates a corresponding peak list for the PXRD pattern shown in FIG. 1

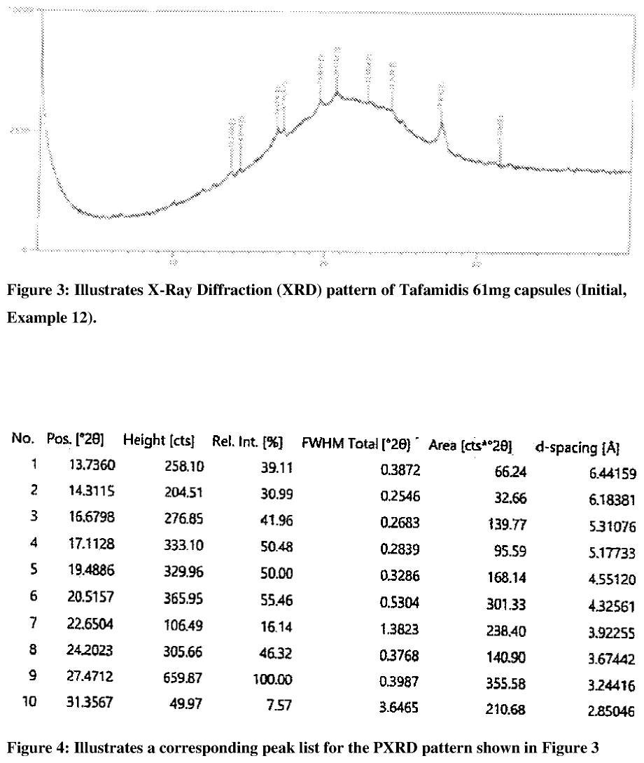

FIG. 3: Illustrates X-Ray Diffraction (XRD) pattern of Tafamidis 61 mg capsules (Initial, Example 12).

FIG. 4: Illustrates a corresponding peak list for the PXRD pattern shown in FIG. 3.

FIG. 5: Illustrates X-Ray Diffraction (XRD) pattern of Tafamidis 61 mg capsules (Stability 40° C./75% RH 6 months, Example 12).

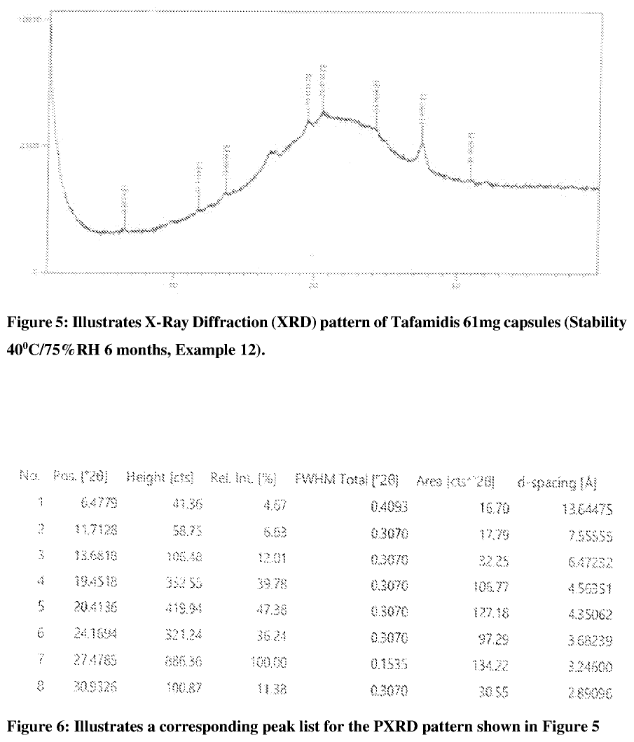

FIG. 6: Illustrates a corresponding peak list for the PXRD pattern shown in FIG. 5

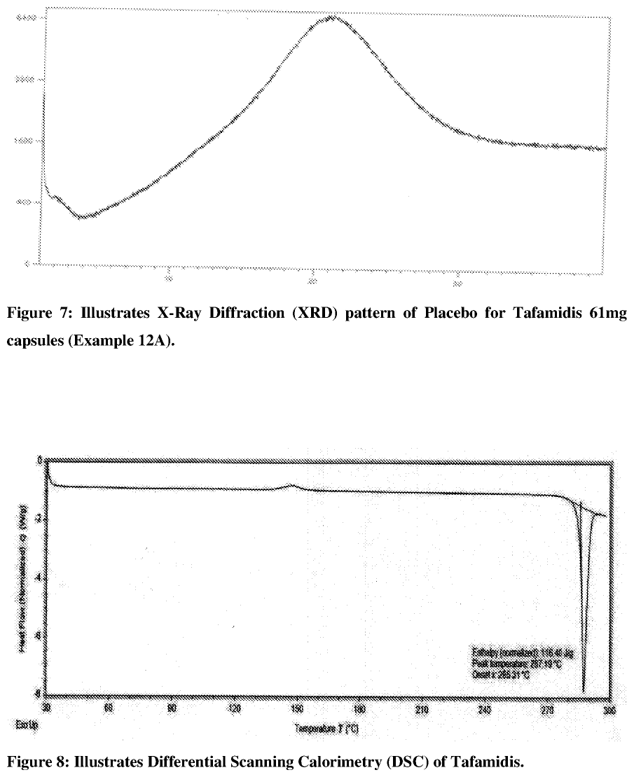

FIG. 7: Illustrates X-Ray Diffraction (XRD) pattern of Placebo for Tafamidis 61 mg capsules (Example 12A).

FIG. 8: illustrates Differential Scanning calorimetry (DSC) of Tafamidis.

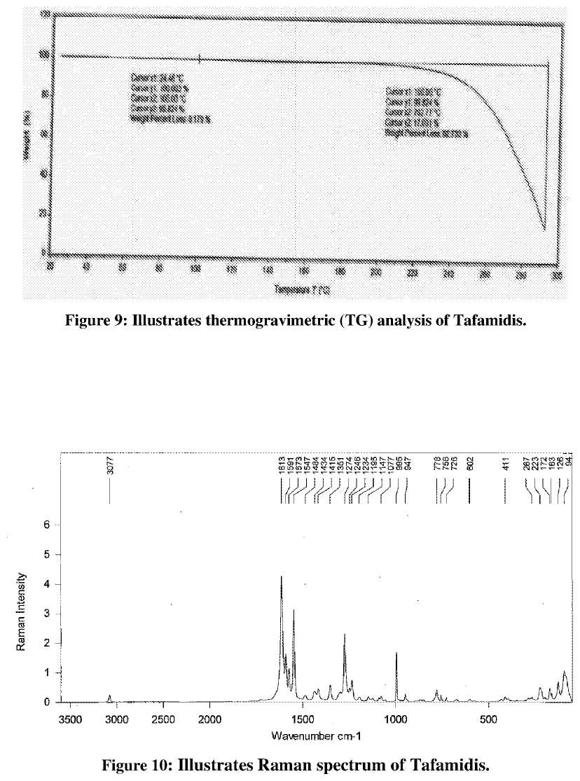

FIG. 9: illustrates thermogravimetric (TG) analysis of Tafamidis.

FIG. 10: illustrates Raman spectrum of Tafamidis.

### DETAILED DESCRIPTION OF THE INVENTION

Definitions of some of the terms used herein are detailed below.

The use of the terms “a” and “an” and “the” and similar references in the context of describing the composition described herein (especially in the context of the following claims) are to be construed to cover both the singular and the plural, unless otherwise indicated herein or clearly contradicted by context.

The “API” as used herein refers to active pharmaceutical ingredient or drug.

The term “about” as used herein embodies standard error associated with a physico-chemical observable. As used herein, the term “about” means a slight variation of the value specified, for example, within 10% of the value specified. A stated amount for a compositional ingredient that is not preceded by the term “about” does not mean that there is no variance for the stated term, as one of ordinary skill would understand that there may be the possibility of a degree of variability generally associated with experimental error.

The term “therapeutically effective amount” or effective dose” as used herein refers to the amount or dose of Tafamidis that is sufficient to initiate therapeutic response in a patient.

The term “treating,” as used herein, unless otherwise indicated, means reversing, alleviating, inhibiting the progress of, or preventing the disorder or condition to which such term applies, or one or more symptoms of such disorder or condition. The term “treatment,” as used herein, unless otherwise indicated, refers to the act of “treating” as defined immediately above.

The term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

As used herein, “stable” as used herein refers to physical stability, chemical stability, and polymorphic stability of a solid oral dosage form, particularly a stable soft gel capsule comprising composition containing Tafamidis Form 6 and at least one or more pharmaceutically acceptable excipients. Physical stability refers to consistent physical properties of composition throughout the product self-life. For e.g., suspension/solution appearance, description, consistency, flow properties, manufacturability, and others. Chemical stability refers to consistency in obtaining acceptable results of drug assay, drug content uniformity and drug related substances. Polymorphic stability refers to retention polymorphic type of Tafamidis API in the finished product and no polymorph conversions throughout the product self-life for e.g., at initial and at accelerated stability conditions (40° C./75% RH 6 months). The different physical properties exhibited by different solid forms of a pharmaceutical compound can affect important pharmaceutical parameters such as storage, stability, compressibility, density (important in formulation and product manufacturing), and dissolution rates (important in determining bioavailability). Stability differences may result from changes in chemical reactivity (e.g., differential hydrolysis or oxidation, such that a dosage form comprising a certain polymorph can discolor more rapidly than a dosage form comprising a different polymorph), mechanical changes (e.g., tablets can crumble on storage as a kinetically favoured crystalline form converts to thermodynamically more stable crystalline form), or both (e.g., tablets of one polymorph can be more susceptible to breakdown at high humidity). Solubility differences between polymorphs may, in extreme situations, result in transitions to crystalline forms that lack potency and/or that are toxic. In addition, the physical properties of a crystalline form may also be important in pharmaceutical processing. Based on a type of polymorph, one cannot predict which polymorph properties is suitable to provide a stable solid oral dosage form of Tafamidis and to methods for their preparation.

In certain embodiments, the present invention provides a process for the preparation of crystalline form of Tafamidis, comprising the steps of:
a. dissolving Tafamidis meglumine (2) in water;
b. adjusting pH of the reaction mass with a suitable acid;
c. adding polar aprotic solvent to the reaction mixture;
d. separating organic layers and adding polar aprotic solvent or mixture of solvents to the organic layer;
e. adding the reaction mixture to suitable solvent.
f. filtering the solid and washing with suitable solvent;
g. milling the wet material at suitable temperature; and
h. drying and isolating Tafamidis.

As used herein the present invention, the organic solvent used is selected from Di tertbutyl ether, diethyl ether, di isopropyl ether, 1,4-dioxane, methyl tert-butyl ether, ethyl tert-butyl ether, tetrahydrofuran and dimethoxy ethane, preferably using tetrahydrofuran; the aprotic solvent is selected from dimethyl sulfoxide, dimethylacetamide, and dimethyl formamide, methylene chloride, ethylene dichloride, carbon tetrachloride or mixtures thereof.

As used herein in step b) “acid” refers to hydrochloric acid, sulphuric acid, and nitric acid, preferably using hydrochloric acid.

In another embodiment of the present invention provides a process for the preparation of crystalline form of Tafamidis (1), comprising the steps of:
i. providing a mixture of Tafamidis in an organic solvent;
ii. heating the reaction mass at suitable temperature;
iii. adding suitable polar aprotic solvent to the reaction mixture;
iv. adding the reaction mixture to suitable aprotic solvent;
v. filtering the solid and milling at suitable temperature; and
vi. drying and isolating Tafamidis.

As used herein the present invention, the organic solvent used is selected from di-tert-butyl ether, diethyl ether, Di isopropyl ether, 1,4-dioxane, methyl tert-butyl ether, ethyl tert-butyl ether, tetrahydrofuran and dimethoxy ethane, preferably using tetrahydrofuran; the polar aprotic solvent is selected from dimethyl sulfoxide, dimethylacetamide, and dimethyl formamide, preferably using dimethylacetamide and the one or more solvent is selected from methylene chloride, ethylene dichloride, carbon tetra chloride, water, ethyl acetate, isopropanol and n-heptane.

In certain embodiments, preparation further comprises washing of Tafamidis. In certain embodiments, washing of Tafamidis comprises washing with a solvent selected from dichloromethane, ethyl acetate, methyl tertiary butyl ether, heptane, or mixtures thereof. In certain embodiments, washing of Tafamidis comprises mixture of antisolvent and solvent. Preferably, the crystals of Tafamidis are washed with dichloromethane.

In certain embodiments, preparation further comprises drying of Tafamidis (1) preferably under reduced pressure, such as under vacuum pressure.

In certain embodiments, Tafamidis process provided in the present invention may involve milling of wet material or optionally partially dried material.

In certain embodiments, Tafamidis process provided in the present invention may involve milling of wet material, which exists as a solvated material.

In another embodiment, Tafamidis obtained in the present invention exists a solvated form. Wherein the solvated form consisting of Dimethyl acetamide, Methylene dichloride, Tetrahydrofuran, ethyl acetate, methanol, or mixture thereof.

In another embodiment, the crystalline form of Tafamidis characterized by a differential scanning calorimetry (DSC) thermogram as the pattern shown in FIG. 8.

In certain embodiment, the crystalline form of Tafamidis characterized by a differential scanning calorimetry (DSC) thermogram comprising an endothermic peak between about 282° C. and about 292° C.

In another embodiment, the crystalline form of Tafamidis characterized by a differential scanning calorimetry (DSC) thermogram comprising an endothermic peak at about 287° C.

In another embodiment, the crystalline form of Tafamidis obtained according to the present invention is having water content less than 5% (w/w), preferably less than 2% (w/w), more preferably less than 1% (w/w).

In certain embodiments, the Thermogravimetric analysis (TGA) of crystalline form of Tafamidis exhibits weight loss of less than 1.0% (w/w) as shown in pattern FIG. 9.

In another embodiment, the crystalline form of Tafamidis is having a specific surface area (SSA) is less than 30 m²/g, more preferably less than 20 m²/g.

In certain embodiments, Tafamidis after milling was checked for X-Ray diffraction pattern and found to be crystalline form 6 having characteristic 2-theta peaks at 13.6±0.2, 20.4±0.2 and 27.5±0.2.

One embodiment of the present invention provides Form 6, wherein said form has a powder X-ray diffraction pattern comprising peaks at diffraction angles (2θ) of 23.8±0.2 and 27.5±0.2. In another embodiment, Form 6 has a powder X-ray diffraction pattern comprising peaks at diffraction angles (2θ) of 13.6±0.2, 23.8±0.2 and 27.5±0.2. In another embodiment, Form 6 has a powder X-ray diffraction pattern comprising peaks at diffraction angles (2θ) of 13.6±0.2, 23.5±0.2, 23.8±0.2, and 27.5±0.2. In another embodiment, Form 6 has a powder X-ray diffraction pattern comprising peaks at diffraction angles (2θ) of 13.6±0.2, 20.4±0.2 and 27.5±0.2.

In another embodiment, Tafamidis (1) obtained in the present invention is characterized by particle size distribution wherein, d₉₀ is less than 100 μm and more preferably less than 50 microns.

In certain embodiment, Tafamidis obtained in the present invention is having total residual solvent content less than 5000 ppm. More preferably less than 3000 ppm and still more preferably less than 1000 ppm.

In another embodiment, Tafamidis having tetrahydrofuran solvent content less than 720 ppm, preferably less than 50 ppm, dichloromethane content less than 600 ppm, preferably less than 197 ppm, and N, N-dimethyl acetamide content less than 1090 ppm, preferably less than 500 ppm. The presence of residual solvents is critical to get the stable crystalline forms.

In another embodiment, the stability of Tafamidis obtained in the present invention may be loaded on stability at accelerated stability at 25±2° C. under 60±5% Relative humidity condition up to 6 months and long-term conditions long term stability at 5±3° C. condition up to 6 months and analysed by X-Ray diffraction pattern of Tafamidis.

In another embodiment, Tafamidis obtained in the present invention is free of nitrosamine impurities.

In another embodiment, a stable solid oral dosage form comprising composition containing Tafamidis Form 6, and at least one or more pharmaceutically acceptable excipients. Solid oral dosage forms include capsules, tablets, dispersions, suspensions, and the like, e.g., enteric-coated capsules and/or tablets, capsules and/or tablets containing enteric-coated pellets containing Tafamidis Form 6, and at least one or more pharmaceutically acceptable excipients.

The compositions may be conveniently presented in unit dosage forms and prepared by any methods known in the pharmaceutical arts. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20™” and “TWEEN 80™”, and Pluronic® F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in Remington: The Science & Practice of Pharmacy, 19th ed., Williams & Williams, (1995), and in the “Physician's Desk Reference”, 52nd ed., Medical Economics, Montvale, N.J. (1998), and in “Handbook of Pharmaceutical Excipients”, 3rd. Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to inhibit transthyretin (TTR) dissociation (i.e. prevents dissociation of the native TTR tetramer into monomers). Compositions will generally contain anywhere from about 0.001% by weight to about 99% by weight active agent, preferably from about 0.01% to about 30% by weight active agent, and more preferably from about 0.01% to 20% by weight active agent and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing granulating and compressing into tablets or dissolving or dispersing the ingredients as appropriate to the desired preparation like soft gel capsule.

The pharmaceutical carrier(s) employed may be either solid or liquid. Exemplary solid carriers include lactose, sucrose, talc, gelatin, agar, pectin, acacia, magnesium stearate, stearic acid, and the like. Exemplary liquid carriers include syrup, peanut oil, olive oil, water, and the like. Similarly, the carrier(s) may include time-delay or time release materials known in the art, such as glyceryl monostearate or glyceryl di stearate alone or with a wax, ethyl cellulose, hydroxy propyl methylcellulose, methyl methacrylate and the like. Suitable liquid vehicles for the invention soft gel dosage forms are fatty oils, liquid paraffin, or liquid polyethylene glycols. suitable liquid vehicle is liquid polyethylene glycols.

For the soft gel dosage forms, the composition contains one or more pharmaceutically acceptable excipients selected from the group consisting of suitable liquid vehicle such as fatty oils, liquid paraffin, or liquid polyethylene glycols; a suitable surfactant such as sodium lauryl sulfate, poloxamer, glyceryl monostearate, glyceryl monolaurate, sorbitan fatty acid esters; a suitable solubilizer such as polyvinylpyrrolidone, a suitable antioxidant Butylated Hydroxytoluene and soft gel capsule shells are comprising of Gelatin, Glycerin, Sorbitol sorbitan, and Purified water. Suitable plasticizers for soft gel capsule shells include glycerin, sorbitol, sorbitol sorbitan and alkylene glycols (e.g., propylene glycol and low molecular weight polyethylene glycols).

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

In another embodiment, a stable solid oral dosage form comprising composition containing Tafamidis Form 6 having d₉₀ particle size less than 100μ, particularly less than 75μ or less than 50μ and at least one or more pharmaceutically acceptable excipients.

In another embodiment, a stable solid oral dosage form comprising composition containing Tafamidis Form 6 having specific surface area less than 30 m²/g, particularly less than 20 m²/g.

In another embodiment, a stable solid oral dosage form comprising composition containing Tafamidis Form 6 having bulk density of 0.20 to 0.50 g/ml, particularly between 0.25 to 0.40 g/ml.

In another embodiment, a stable soft gel capsule comprising Tafamidis Form 6 having d₉₀ less than 100μ, Polysorbate 20, Povidone K-30, Butylated hydroxytoluene and Polyethylene glycol 400.

In another embodiment, a stable soft gel capsule comprising Tafamidis Form 6 having d₉₀ less than 50μ, Polysorbate 20, Povidone K-30, Butylated hydroxytoluene and Polyethylene glycol 400.

In another embodiment, a stable solid dosage form composition comprising composition containing Tafamidis is indicated for the treatment of the cardiomyopathy of wild type or hereditary transthyretin-mediated amyloidosis in adults to reduce cardiovascular mortality and cardiovascular-related hospitalization.

### Method of Analysis:

In another embodiment, the crystalline form of Tafamidis obtained by any of the above methods were analysed using X-ray powder diffractometer and characterized by the X-ray powder diffraction graphs as shown in FIG. 1 with below mentioned operating parameters.

### Operation Parameters:

| Name of Instrument | D8 Advance, Bruker Corporation, Germany |
|---|---|
| Wavelength | λ = 1.54056 A using Cu—Kn radiation |
| Detector | Scintillation counter |
| Voltage (kV), Current (mA) | 40, 35 |
| Anode | Copper |
| Scan Type, Scan Mode | Locked, Coupled |
| Angular Range (°2 | 4 to 6 |
| Step size (°) | 0.0050 |
| Time per Step (Seconds) | 6 |

### Raman Spectroscopy: Raman Spectra are Recorded on a Bruker Multiram FT

Raman system with below mentioned operating parameters.

| Actual laser wave number | 1064 | cm⁻¹ |
|---|---|---|
| Raman Layer Power (mW) | 300 | |
| Scan time or Scans | 3 | min |

### Specific Surface Area: Specific Surface Area is Analysis by USP Method.

| | |
|---|---|
| Instrument Name | Surface Area Analyzer |
| Make | Smart Sorb |
| Model | 92/93 |
| N2 Partial Pressure % | 29.90 |
| Analysis method | BET Single Point Dynamic Method |
| Adsorbate Gas | Nitrogen |

The information that follows illustrates various embodiments of the compositions disclosed herein. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and also combined with other important aspects of the invention described elsewhere within this specification as a whole and including the examples and figures.

The best mode of carrying out the present invention is illustrated by the below mentioned examples. These examples are provided as illustration only and hence should not be construed as limitation to the scope of the invention. The skilled person will appreciate that the compositions claimed herein extends to such combinations of features and that these have not been recited in detail here in the interests of brevity.

### EXAMPLES

The following examples are provided to illustrate the present invention. It should be understood, however, that the invention is not to be limited to the specific conditions or details described in these examples.

### Example-1: Preparation of Tafamidis from Tafamidis Meglumine

To 100 g of Tafamidis meglumine (2), 10 volumes of DM water were added at 25 to 30° C. and stirred for 10-15 minutes. The reaction mass was acidified with 50% hydrochloric acid solution to adjust pH 6.0 to 6.5 and stirred for 10 to 20 minutes. 40 volumes of Tetrahydrofuran were added to the reaction mass at 25 to 30° C. The reaction mass temperature was maintained at 55 to 60° C. and stirred for 10-15 minutes. The organic and aqueous layer were separated and 100 ml of N, N-dimethyl acetamide was added to the organic layer at 25 to 30° C. The reaction mass was added to 100 volumes of dichloromethane at 0-5° C. and stirred. The obtained solid was filtered and washed with 100 ml of dichloromethane. The solid was dried and milled to get the titled compound. Yield: 35 g. XRD: FIG. 1; Purity: >99.5

### Example-2: Preparation of Tafamidis from Tafamidis Meglumine

To 100 g of Tafamidis meglumine (2), 10 volumes of DM water were added at 25 to 30° C. and stirred for 10-15 minutes. The reaction mass was acidified with 50% hydrochloric acid solution to adjust pH 6.0 to 7.0 and stirred for 10 to 20 minutes. 40 volumes of Tetrahydrofuran were added to the reaction mass at 25 to 30° C. The reaction mass temperature was maintained at 55 to 60° C. and stirred for 1 hr. The organic and aqueous layer were separated and 1:1 mixture of 100 ml of N, N-dimethyl acetamide and dichloromethane was added. The obtained solid was washed with 100 ml of dichloromethane, dried and micronized to get the titled compound.

Yield: 40 g. XRD: FIG. 1; Purity: >99.5

### Example-3: Preparation of Tafamidis

A mixture of Tafamidis (50 g) and tetrahydrofuran (600 ml) was stirred for 5 minutes at 25-30° C. The reaction mixture was heated to 60-65° C. and stirred for 10 min at the same temperature. Dimethyl acetamide (100 ml) was added to the reaction mixture at 60-65° C. and stirred for 30 minutes. The reaction mixture was added to methylene dichloride (1.5 L) at 0-5° C. and stirred for 5 hours at the same temperature. The precipitated solid was filtered and washed with methylene dichloride. Methylene dichloride (500 ml) was added to the obtained wet material and stirred for 2 hours at 25-30° C. The obtained solid was filtered under vacuum and washed with methylene dichloride then milled. The final solid was dried get the title compound. Yield: 60%; Purity: >99.5.

### Example-4: Preparation of Tafamidis Dimethylacetamide Solvate

To 100 g of Tafamidis meglumine (2), 15 volumes of DM water were added to at 25 to 30° C. and stirred for 30 minutes. The reaction mass pH was adjusted to 6.0 to 7.0 and stirred for 30 minutes. 50 volumes of Tetrahydrofuran were added to the reaction mass at 25 to 30° C. The reaction mass temperature was maintained at 60° C. for 1 hr. The organic and aqueous layer were separated, and the organic layer was added to 100 ml of dimethyl acetamide at 60-65° C. and stirred for 30 minutes. The reaction mixture was added to 500 ml of dichloromethane at 0-5° C. The obtained solid was dried and to get the titled compound. Yield: 40 g. Purity: >99.5.

### Example-5: Preparation of Tafamidis Dichloromethane Solvate

A mixture of Tafamidis (50 g) and tetrahydrofuran (600 ml) was stirred for 20 minutes at 25-30° C. The reaction mixture was heated to 60-65° C. and stirred for 30 min. Dimethyl acetamide (100 ml) was added to the reaction mixture at 60-65° C. and stirred for 30 minutes. The obtained solution was added to 500 ml of methylene dichloride at 0-5° C. and stirred for 5 hours. The reaction mass was filtered, washed the material with methylene dichloride and dried. Methylene dichloride (500 ml) was added to the obtained wet material at 25-30° C. and stirred for 2 hours. The solid was filtered and dried for 12 hrs to get the title compound. Yield: 60%; Purity: >99.5.

### Example-6: Preparation of Tafamidis Ethyl Acetate Solvate

A mixture of Tafamidis (50 g) and tetrahydrofuran (600 ml) was stirred for 20 minutes at 25-30° C. The reaction mixture was heated to 60-65° C. and stirred for 30 min. Dimethyl acetamide (100 ml) was added to the reaction mixture at 60-65° C. and stirred for 30 minutes. The obtained solution was added to methylene dichloride at 0-5° C. and stirred for 5 hours. The reaction mass was filtered, stirred in a mixture of 5% ethyl acetate and methylene dichloride (500 ml) at 25-30° C. for 2 hours. The solid was filtered and dried to get the title compound. Yield: 60%; Purity: >99.5.

### Example-7: Preparation of Tafamidis Isopropyl Alcohol Solvate

A mixture of Tafamidis (50 g) and tetrahydrofuran (600 ml) was stirred for 20 minutes at 25-30° C. The reaction mixture was heated to 60-65° C. and stirred for 30 min. Dimethyl acetamide (100 ml) was added to the reaction mixture at 60-65° C. and stirred for 30 minutes. The obtained solution was added to methylene dichloride at 0-5° C. and stirred for 5 hours. The reaction mass was filtered, stirred in a mixture of 5% Isopropyl alcohol and methylene dichloride (500 ml) at 25-30° C. for 2 hours. The solid was filtered and dried to get the title compound. Yield: 50%; Purity: >99.5.

### Examples 8-9: Soft Gel Capsule Formulation Containing Tafamidis Form 6 with Different Solubilizing Agents

| | | Example 8 | Example 9 |
|---|---|---|---|
| S. | | Quantity | Quantity |
| No | Name of Ingredient | (% w/w) | (% w/w) |
| 1 | Tafamidis Form 6 | 10.00% | 10.00% |
| 2 | Polysorbate 20 | 9.25% | 9.25% |
| 3 | Povidone K-30 | 2.00% | — |
| 4 | Povidone K-90 | — | 2.00% |
| 5 | Butylated Hydroxytoluene | 0.23% | 0.23% |
| 6 | Polyethylene Glycol 400 | 78.52% | 78.52% |

| | | |
|---|---|---|
| Fill weight of capsule (% w/w) | 100% | 100% |

### Process of Preparation:

### Capsule Fill Preparation Process:

1. Majority Batch quantity of Polyethylene Glycol 400 were taken into mixing tank.

2. A clear solution was obtained by adding batch quantity of Butylated Hydroxytoluene to step-1 and mixing.

3. A clear solution was obtained by adding batch quantity of Povidone to step-2 and mixing.

4. A clear solution was obtained by adding Polysorbate 20 to step-3 solution under stirring and then adding remaining batch quantity of Polyethylene Glycol 400.

6. A suspension was prepared by adding Tafamidis Form 6 API to step-4 and added remaining batch quantity of Polyethylene Glycol 400. Mixing continued in mixer tank under constant stirring.

7. The final suspension/fill was collected into product fill mixing tank.

8. The final suspension was mixed well under constant stirring and deaerated by applying vacuum.

### Capsule Shell Preparation Process:

1. Hot water circulation was allowed in Gelatin melting tank.

2. Batch quantities of Glycerin, Sorbitol sorbitan solution and Purified water were passed through suitable screen/mesh and mixed well.

3. Batch quantity of Gelatin was added into gelatin melting tank and mixed well until creamy mixture was formed.

4. Red iron oxide and glycerin were milled together in colloid mill and mixed well in gelatin mixing tank till uniform colour mixing was observed.

5. Gelatin mass was unloaded and filtered through suitable mesh into gelatin mass holding tank and temperature was maintained at 55±5° C.

6. Red iron oxide and glycerin were milled together in colloid mill and added into gelatin melting tank with continuous stirring.

7. Vacuum was applied to gelatin mass. Further, gelatin mass was examined for the presence of air bubbles and homogeneity of gelatin mass. If air bubble observed vacuum applied till satisfactory gelatin mass was obtained.

8. Gelatin mass was unloaded and filtered through suitable mesh into gelatin mass holding tank and temperature was maintained at 55±5° C.

### Encapsulation:

1. Nitrogen purging was maintained in medicine hopper throughout the encapsulation process.

2. Fill weight was checked by using fill medicament.

3. The injection was started by the shut off valve over the wedge and the fill material was allowed to be injected into the cavities to form the capsules which fell on the conveyor and the capsules passed into the tumble drier.

4. Filled weights were checked for every one-hour interval for all cavities and recorded.

### Drying of Capsules:

1. Capsules were collected from the tumble drier and spreaded into trays and kept in capsule drying area.

2. Hardness of capsules were checked for every one-hour till the desired hardness was not more than 10N.

3. After achieving desired hardness, the average hardness and gross weight were recorded.

4. The capsules were kept under drying and hardness monitored to avoid over drying of capsules.

Example 8 & 9 Tafamidis capsules were subjected to dissolution testing under dissolution conditions of 0.05M Sodium Phosphate, pH 6.8 with 1% Tween 80, Type II USP (paddle) with sinker, 75 RPM, 900 mL at temperature of 37° C.±0.5° C. The dissolution data on two Tafamidis formulations indicated that the batch manufactured with Povidone K-30 resulted in the slightly faster profile as compared to Povidone K-90. It was concluded that not less than 75% of Tafamidis released at 45 minutes in Example-8 Povidone K-30 formulation.

### Examples 10-11: Soft Gel Capsule Fill of Example 8 was Encapsulated into Gelatin Shell Prepared with Two Different Gelatin, Type A & B

| S. | | Example 10 | Example 11 |
|---|---|---|---|
| No | Name of Ingredient | Quantity (% w/w) | Quantity (% w/w) |
| 1 | Gelatin Type B | 40.20% | — |
| 2 | Gelatin Type A | — | 40.20% |
| 3 | Glycerin | 4.55% to 9.54% | 4.55% to 9.54% |
| 4 | Sorbitol sorbitan solution | 9.53% to 14.52% | 9.53% to 14.52% |
| 5 | Red iron oxide | 0.47% | 0.47% |
| 6 | Purified water | Q.s | Q.s |

| | | |
|---|---|---|
| Total weight | 100.00% | 100.00% |

Q.s: Quantity sufficient

Example 10 & 11 Tafamidis capsules were subjected to dissolution testing under dissolution conditions of 0.05M Sodium Phosphate, pH 6.8 with 1% Tween 80, Type II USP (paddle) with sinker, 75 RPM, 900 mL at temperature of 37° C.±0.5° C. The dissolution data on two Tafamidis formulations indicated that the batch manufactured with Type B gelatin resulted in the slightly faster profile as compared to Type A. It was concluded that not less than 75% of Tafamidis released at 45 minutes in Example-10 Type B gelatin formulation.

Different trials carried out with different ratios of plasticizers:—Based on the initial development studies, Type B gelatin was selected, and the batches were executed to optimize the ratio of two plasticizers in which Glycerin is one of these two plasticizers. In one of the trials two plasticizers taken were Glycerin and Sorbitol sorbitan solution in different ratios of Glycerin to Sorbitol sorbitan solution i.e., 40:60 and 50:50 ratios. Based on the data generated on chemical and physical characteristics of the capsules it was concluded that the manufacturing process resulted in capsules without weight variations and hardness. The dissolution data was acceptable and not less than 75% of Tafamidis was released in 45 minutes when there 40:60 and 50:50 ratios of the Glycerin to Sorbitol sorbitan solution. The Tafamidis dissolution data was not acceptable when ratio of two plasticizers is different than 40:60 and/or 50:50.

### Examples 12 & 12A (Placebo): Soft Gel Capsule Formulation Containing Tafamidis Form 6 with d₉₀ Less than 100 μm, Bulk Density of 0.20 to 0.50 g/ml, Specific Surface Area of Less than 30 m²/g

| | | | Example 12A |
|---|---|---|---|
| | | Example 12 | (Placebo) |
| S. | | Quantity | Quantity |
| No | Name of Ingredient | (% w/w) | (% w/w) |
| 1 | Tafamidis Form 6 | 10.20% | — |
| 2 | Polysorbate 20 | 9.25% | 9.25% |
| 3 | Povidone K-30 | 2.00% | 2.00% |
| 4 | Butylated Hydroxytoluene | 0.07% | 0.07% |
| 5 | Polyethylene Glycol 400 | 78.48% | 88.68% |

| | | |
|---|---|---|
| Fill weight of capsule (mg) | 100.00% | 100.00% |

The brief manufacturing process of examples 12 & 12A were same as outlined above under example 8 & 9.

Chemical Characteristics of the Capsules after Drying:

| Test | Example 12 |
|---|---|
| Assay by HPLC (% w/w) | 95% to 105% |
| Water content of capsule fill | Less than 15% |
| composition (% w/w) | |
| Hardness of soft gel capsule | Not more than 10N |
| Content uniformity (% w/w) by | Within the acceptance limits |
| HPLC | of USP <905> |
| Related Substances (% w/w) | Within the ICH limits |
| KSM-I Impurity | |
| Stage-I Impurity | |
| Mono chloro Tafamidis impurity | |
| Single maximum unspecified | |
| impurity | |
| Total impurities | |

Based on the data generated on physical and chemical characteristics of the Example 12 Tafamidis capsules it was concluded that the manufacturing process resulted in capsules without weight variations and hardness.

The drug assay, drug content uniformity, related substances were within the acceptable drug regulatory limits. Further, the drug dissolution profiles of example 12 formulation were comparable to VYNDAMAX (Tafamidis 61 mg capsules). Therefore, it was concluded that Tafamidis Form 6 having d₉₀ particle size less than 100μ was promising and unexpected.

Stability testing of Example 12 (Tafamidis 61 mg capsules): Capsules were loaded onto accelerated stability testing (40° C./75% RH) for 6 months. Physical and chemical characteristics were satisfactory and within the acceptable limits. Further, polymorphic characterization was carried out on input Form 6 API, initial samples (example 12), placebo capsules (example 12A) and stability samples (example 12) and as indicated in FIGS. 1-7 Form 6 got retained in the capsule with no polymorph conversions.

Bioequivalence testing was carried out under Fasting and Fed conditions on invention Tafamidis 61 mg soft gel capsule containing Form 6 against VYNDAMAX 61 mg capsules, it was concluded that invention Tafamidis Form 6 soft gel capsule formulation was bioequivalent to VYNDAMAX 61 mg capsules with respect to pharmacokinetic parameters of Cₘₐₓ and AUC₀₋ₜ. Upon oral administration of the Tafamidis capsule to healthy adult subjects under fasting and fed conditions results in a mean Cₘₐₓ of about 4000 ng/ml to about 5000 ng/ml and mean AUC₀₋ₜ is about 130000 ng·hr/ml to about 150000 ng·hr/ml.

### Example 13: Hard Gelatin Capsule Formulation Containing Tafamidis with d₉₀ Less than 100 μm, Bulk Density of 0.20 to 0.50 g/Ml, Specific Surface Area of Less than 30 m²/g

| | | Example 13 |
|---|---|---|
| S. | | Quantity |
| No | Name of Ingredient | (% w/w) |
| 1 | Tafamidis | 15% |
| 2 | Povidone K30 | 8% |
| 3 | Polyethylene glycol 6000 PEG | 15% |
| 4 | Purified water | Quantity |
| | | sufficient |
| 5 | Lactose monohydrate | 57% |
| 6 | Crospovidone | 4% |
| 7 | Colloidal silicon dioxide | 1% |

| | |
|---|---|
| Capsule fill weight % w/w | 100.00% |

Brief manufacturing process: Batch quantity of Povidone and PEG was added into purified water and mixed well to obtain granulating fluid. Batch quantity of lactose monohydrate and Tafamidis were sifted and then granulated with Povidone+PEG solution in a Fluid bed granulator (Top-spray granulation). The dried granules were blended with extra granular excipients (Crospovidone and Colloidal silicon dioxide).

Upon dissolution testing it was concluded that not less than 75% of Tafamidis released in 45 minutes.

### Examples 14: Tablet Formulation Containing Tafamidis with d₉₀ Less than 100 μm, Bulk Density of 0.20 to 0.50 g/Ml, Specific Surface Area of Less than 30 m²/g

| | | Example 14 |
|---|---|---|
| S. | | Quantity |
| No | Name of Ingredient | (% w/w) |
| 1 | Tafamidis | 15% |
| 2 | Povidone K30 | 5% |
| 3 | Polyethylene glycol 6000 (PEG) | 15% |
| 4 | Purified water | Quantity |
| | | sufficient |
| 5 | Microcrystalline cellulose | 30.5% |
| 6 | Lactose monohydrate | 30% |
| 7 | Crospovidone | 3% |
| 8 | Colloidal silicon dioxide | 1% |
| 9 | Magnesium stearate | 0.5% |

| | |
|---|---|
| Tablet weight % w/w | 100.00% |

Brief manufacturing process: Batch quantity of Povidone and PEG was added into purified water and mixed well. Batch quantity of Microcrystalline cellulose, Lactose monohydrate and Tafamidis were sifted and then granulated with Povidone+PEG solution in a Fluid bed granulator (Top-spray granulation). The dried granules were blended with extra granular excipients (Crospovidone and Colloidal silicon dioxide). The blend was lubricated with magnesium stearate and compressed into tablets.

Upon dissolution testing it was concluded that not less than 75% of Tafamidis released in 45 minutes.

1. (canceled)

2. (canceled)

3. A process for the preparation of Tafamidis, comprising the steps of:
a. dissolving Tafamidis meglumine (2) in water;
b. adjusting pH of the reaction mass with a suitable acid;
c. adding polar aprotic solvent to the reaction mixture;
d. separating organic layers and adding polar aprotic solvent or mixture of solvents to the organic layer;
e. adding the reaction mixture to suitable solvent;
f. filtering the solid and washing with suitable solvent;
g. milling the wet material at suitable temperature; and
h. drying and isolating Tafamidis

4. A process for the preparation of Tafamidis, comprising the steps of:
a. providing a mixture of Tafamidis in an organic solvent;
b. heating the reaction mass at suitable temperature;
c. adding suitable polar aprotic solvent to the reaction mixture;
d. adding the reaction mixture to suitable solvent;
e. filtering the solid and washing with suitable solvents;
f. milling the wet material at suitable temperature; and
g. drying and isolating Tafamidis.

5. The process as claimed in claims 3 and 4, wherein the organic solvent is selected from selected from di-tert-butyl ether, diethyl ether, Di isopropyl ether, 1,4-dioxane, methyl tert-butyl ether, ethyl tert-butyl ether, tetrahydrofuran, and dimethoxy ethane; wherein the suitable polar aprotic solvent is selected from dimethyl sulfoxide, dimethylacetamide, and dimethyl formamide; methylene chloride, ethylene dichloride, carbon tetrachloride, water, ethyl acetate, isopropanol. and n-heptane or mixtures thereof.

6. The process as claimed in claims 3 and 4, wherein Tafamidis is having water content less than 1% (w/w).

7. The process as claimed in claims 3 and 4, wherein the drying step is performed under vacuum at temperature 60-65° C.

8. The process as claimed in claims 3 and 4, wherein Tafamidis is free of nitrosamine impurities.

9. The process as claimed in claims 3 and 4, wherein said Tafamidis has total residual content less than 3000 ppm.

10. A process for the preparation of Tafamidis, comprising the steps of:
a) milling wet material of Tafamidis solvate at suitable temperature;
b) drying the material to remove residual solvent;
wherein the Tafamidis obtained in step b) consists of at least one of the properties selected from a group consisting of:
i. moisture content less than 1% (w/w);
ii. d₉₀ particle size of less than 100 microns;
iii. specific surface area of less than 30 sq·m/g; and
iv. total residual solvents less than 3000 ppm.

11.-30. (canceled)
